(19) United States
(12) Patent Application Publication
(10) Pub. No.: US 2025/0345022 A1
(43) Pub. Date: Nov. 13, 2025

(54) A PHONOCARDIOGRAM SENSING DEVICE

(71) Applicant: Ticking Heart Pty Ltd, Mount Lawley (AU)

(72) Inventors: Kevin Fynn, Mount Lawley (AU); David Ward, Stonefille (AU); Sven Nordholm, Mount Lawley (AU); Teng-Wen Chang, Taichung City (TW); Matthew Fynn, Mount Lawley (AU); Wei-Juet Wong, Willetton (AU); Hooi Lit NG, Winthrop (AU); Kent Gibson, Noranda (AU); John Siliquini, Maylands (AU); Jeffery Hughes, Highgate (AU); Kayapanda Mandanna, Kolkata (IN)

(73) Assignee: Ticking Heart Pty Ltd, Mount Lawley (AU)

(21) Appl. No.: 18/876,584

(22) PCT Filed: Jun. 18, 2023
(86) PCT No.: PCT/AU2023/050541

§ 371 Date: Dec. 18, 2024

(30) Foreign Application Priority Data
Jun. 24, 2022 (AU) 2022901768

(51) Int. Cl. A61B 7/00; A61B 5/00; A61B 5/0205; A61B 5/021; A61B 5/024; A61B 5/08; A61B 5/1455; A61B 5/282; A61B 5/352; A61B 5/353; A61B 5/355; A61B 7/04; G16H 50/20

(52) U.S. Cl. CPC A61B 7/003; A61B 5/0205; A61B 5/021; A61B 5/02405; A61B 5/0816; A61B 5/14551; A61B 5/282; A61B 5/352; A61B 5/353; A61B 5/355; A61B 5/4878; A61B 5/7203; A61B 7/04; G16H 50/20; A61B 2560/0247; A61B 2560/0468; A61B 2562/0204

(57) ABSTRACT

A phonocardiogram (PCG) sensing device is disclosed that comprises a body audio sensing device including a body audio sensing transducer arranged to sense animal body audio when the body audio sensing device is disposed adjacent an animal body in a body audio sensing position and an acoustic path for body audio is defined between the animal body and the body audio sensing transducer. The PCG sensing device also includes an ambient audio sensing device including an ambient audio sensing transducer arranged to sense ambient audio present in an environment adjacent the phonocardiogram sensing device when the body audio sensing device is disposed in the body audio sensing position. The body audio sensing device produces a body audio signal indicative of animal body audio, the ambient audio sensing device produces an ambient audio signal indicative of the ambient audio, and the ambient audio signal is used to increase the signal to noise ratio of the body audio signal.

### FIELD OF THE INVENTION

The present invention relates to a phonocardiogram sensing device, to a monitoring system for monitoring body generated data, and to a wearable component for a monitoring system.

### BACKGROUND OF THE INVENTION

Cardiovascular disease (CVD) is the leading cause of death worldwide. CVD typically develops gradually, usually over many years, to the extent that very small changes may occur each year and clear symptoms may not develop until the disease has progressed for many years.

An initial step in evaluating whether CVD exists in a patient's heart involves listening to heart sounds-referred to as heart auscultation—as such sounds provide important initial clues in patient evaluation and serve as a guide for further diagnostic testing.

Heart sounds are generated by opening and closing of heart valves and movement of blood through the heart and adjacent vessels. The main normal heart sounds, referred to as S1 and S2, are sounds that correspond to closure of particular heart valves. Abnormalities in the S1 and S2 sounds can be indicative of a heart anomaly. In addition, the presence of an S3 sound may indicate an anomaly caused by disease, and an S4 sound may indicate a pathological condition.

However, heart sounds are difficult to interpret by a clinician, especially for a clinician that is inexperienced at heart auscultation. While an experienced clinician may be able to determine some heart conditions based on intensity, frequency, location and timing of the cardiac cycle, there are significant limitations since successful detection of an anomaly depends on the clinician's skill and many relevant heart sounds have frequencies below the range of human hearing.

Based on a patient examination, a decision is made by a clinician as to whether further investigation is required. If risk factors or indicators identified at the examination indicate that a heart related anomaly may exist, an electrocardiogram (ECG) may be carried out in order to obtain additional information in relation to heart functionality.

An electrocardiogram (ECG) records the electrical activity associated with blood flow through the heart, lungs and other organs during a heartbeat cycle. Using electrodes placed on the skin, small electrical signals can be detected that are a consequence of cardiac muscle depolarisation and repolarisation. During a cardiac cycle, a normal ECG pattern consists of a number of components—a P wave which represents depolarisation of the atria, a QRS complex which represents the depolarisation of the ventricles, and a T wave which represents repolarisation of the ventricles.

During a cardiac cycle, a healthy heart produces a substantially consistent and repeatable pattern, and an ECG recording provides information about the heart rate and rhythm, including information that may be indicative of heart anomalies such as arrhythmia, enlargement of the heart due to hypertension, and myocardial infarction.

Other diseases are also detectible by auscultation. For example, sounds generated by lungs may indicate a lung abnormality, including diseases such as pneumonia, emphysema, asthma, bronchitis and cancer. Such sounds may include:
rhonchi—which are low pitched crackles that can be caused by bronchial tubes containing fluid or mucus;
wheezing—which is a shrill whistle indicating a partially blocked airway; and
stridor—which is a high-pitched turbulent sound usually indicating an obstruction or narrowing in an upper airway.

However, as with heart auscultation, lung auscultation also has significant limitations since successful detection of an anomaly requires the relevant indicative sounds to be audible and accurate diagnosis depends on the clinician's skill.

### SUMMARY OF THE INVENTION

In accordance with a first aspect of the present invention, there is provided a phonocardiogram (PCG) sensing device comprising:
a body audio sensing device including a body audio sensing transducer arranged to sense animal body audio when the body audio sensing device is disposed adjacent an animal body in a body audio sensing position, wherein an acoustic path for body audio is defined between the animal body and the body audio sensing transducer when the body audio sensing device is disposed in the body audio sensing position; and
an ambient audio sensing device including an ambient audio sensing transducer arranged to sense ambient audio present in an environment adjacent the phonocardiogram sensing device when the body audio sensing device is disposed in the body audio sensing position;
the body audio sensing device producing a body audio signal indicative of animal body audio, and the ambient audio sensing device producing an ambient audio signal indicative of the ambient audio, the ambient audio signal usable to increase the signal to noise ratio of the body audio signal.

In an embodiment, the PCG sensing device includes a diaphragm that defines an audio cavity when the body audio sensing device is disposed adjacent the animal body, the diaphragm enhancing body sounds emanating from the animal body.

In an embodiment, the body audio sensing device is connected to the ambient audio sensing device using a vibration reducing connection. The vibration reducing connection may include a resilient O-ring received in opposed circumferential grooves when the body audio sensing device is connected to the ambient audio sensing device.

In an embodiment, the body audio sensing transducer comprises a MEMS microphone.

In an embodiment, the body audio sensing device includes PCG filtering components arranged to filter body audio signals produced by the body audio sensing transducer.

In an embodiment, the body audio sensing device includes a PCG amplification component arranged to amplify body audio signals produced by the body audio sensing transducer. The PCG amplification component may have an associated gain of about 5.

In an embodiment, the ambient audio sensing transducer comprises a MEMS microphone.

In an embodiment, the ambient audio sensing device includes ambient filtering components arranged to filter ambient audio signals produced by the ambient audio sensing transducer.

In an embodiment, the ambient audio sensing device includes an ambient amplification component arranged to amplify ambient audio signals produced by the ambient audio sensing transducer. The ambient amplification component may have an associated gain of about 20.

In accordance with a second aspect of the present invention, there is provided a wearable garment including at least one PCG sensing device according to the first aspect of the present invention.

In an embodiment, the wearable garment comprises a plurality of PCG sensing devices disposed at locations selected to optimise collection of relevant body audio signals.

In an embodiment, the plurality of PCG sensing devices comprises a front plurality of PCG devices that includes PCG devices disposed during use on opposite sides of a sternum of the animal body in alignment with a pulmonary artery, PCG devices disposed during use on opposite sides of the sternum of the animal body in alignment with a tricuspid valve, a PCG device disposed during use adjacent a mitral area on the midclavicular line, and a PCG device disposed during use adjacent a midaxillary area on the midaxillary line.

In an embodiment, the plurality of PCG sensing devices comprises a rear plurality of PCG devices that includes PCG devices disposed on opposite sides of a vertical centreline just below a scapula of the animal body, PCG devices disposed on opposite sides of the vertical centreline at a middle portion of the back of the animal body, and PCG devices disposed on opposite sides of the vertical centreline adjacent lower lobes of the lungs of the animal body.

In an embodiment, the plurality of PCG sensing devices comprises at least one neck PCG device disposed at a patient's neck area to sense audio from the carotid artery.

In an embodiment, the wearable garment comprises a plurality of ECG electrodes disposed at locations selected to optimise collection of relevant body electrical signals.

In an embodiment, the plurality of ECG electrodes comprises a RA ECG electrode disposed rightwardly of an atria of the animal body, and a LA ECG electrode disposed leftwardly of the atria.

In an embodiment, the plurality of ECG electrodes includes a RLD ECG electrode disposed vertically in alignment with the RA ECG electrode at a location below the heart of the animal body.

In an embodiment, the wearable garment includes a right leg drive (RLD) amplifier that uses signals obtained from the RA and LA electrodes to drive a feedback electrical signal into the animal body at the RLD electrode, the feedback signal serving to improve the common mode rejection by counteracting a common mode signal that would otherwise be present in the RA and LA electrodes.

In an embodiment, the wearable garment includes at least one ultrasound sensor.

In an embodiment, information from the at least one ultrasound sensor is used to obtain respiratory and heart cycle data.

In an embodiment, the at least one ultrasound sensor is disposed on the wearable device such that during use the at least one ultrasound sensor is disposed adjacent a patient's heart or thorax.

In an embodiment, the wearable garment includes at least one photoplethysmography (PPG) sensor.

In an embodiment, data from the at least one photoplethysmography (PPG) sensor is used to monitor changes in blood volume and blood oxygenation.

In an embodiment, data from the at least one photoplethysmography (PPG) sensor is used to derive physiological parameters associated with heart rate variability, blood pressure, ankle-brachial pressure, cardiovascular disease, aging, neurological disorder, lung disease and/or respiratory rate.

In an embodiment, data obtained from the at least one ultrasound sensor and data derived from the at least one PPG sensor are used to detect the presence of fluid in a patient's lungs.

In an embodiment, the wearable garment includes a data collection device arranged to synchronously receive signals from the PCG devices and the ECG electrodes.

In an embodiment, the data collection device is arranged to synchronously receive signals from the at least one ultrasound device, the PCG devices and the ECG electrodes.

In an embodiment, the data collection device is arranged to synchronously receive signals from the at least one PPG device, the at least one ultrasound device, the PCG devices and the ECG electrodes.

In an embodiment, the data collection device includes a PCG signal filtering stage arranged to filter PCG signals received from the PCG devices.

In an embodiment, the PCG signal filtering stage includes an AC coupling filter arranged to remove a DC offset voltage in a PCG signal.

In an embodiment, the data collection device includes an instrumentation amplifier that receives ECG signals from the RA and LA ECG electrodes and amplifies a difference between the RA and LA ECG signals.

In an embodiment, the data collection device includes a DC blocking amplifier arranged to remove DC components from an amplified signal produced by the instrumentation amplifier.

In an embodiment, the data collection device includes a notch filter that may be arranged to suppress interference at about 50 Hz.

In an embodiment, the data collection device includes an A/D converter arranged to convert analogue PCG and ECG signals to digital PCG and ECG signals. The A/D converter may be a sigma-delta A/D converter.

In an embodiment, the PCG signal filtering stage includes an antialiasing filter arranged to ensure that substantially no aliasing occurs in the A/D converter.

In an embodiment, the wearable garment includes a memory arranged to store data indicative of PCG and ECG signals.

In an embodiment, the wearable garment includes a wireless transceiver arranged to facilitate communication of data indicative of the PCG and ECG signals from the wearable device.

In accordance with a third aspect of the present invention, there is provided a system for monitoring body generated data, the system comprising:
a wearable device according to the second aspect of the present invention;
a noise cancelling component arranged to use the ambient audio signal to remove ambient associated noise from the body audio signal and thereby produce a noise processed body audio signal having an increased signal to noise ratio; and
a feature extractor arranged to extract a plurality of features from the noise processed body audio signal and to produce at least one feature vector comprising a plurality of the features; and
a data analysis component arranged to use the at least one feature vector to predict at least one medical anomaly associated with the animal body.

In an embodiment, the data analysis component uses at least one machine learning component trained to learn relationships between the feature vectors and medical diagnoses.

In an embodiment, the extracted features are associated with an ECG signal and are derived using R peaks, P peaks, Q points, S-points and T-peaks of the ECG signal, the extracted features derived using a time interval between R peaks, a difference between successive R peak time intervals, and/or a time between P and R peaks.

The extracted features associated with an ECG signal may be obtained using:
frequency domain properties that may include average, variance and higher order moments; skewness; kurtosis; and/or
time domain and time-frequency domain properties that may be obtained using wavelets or wavelet packets (WP) and/or synchronised wavelet packets (SWP) of the ECG signal.

In an embodiment, the extracted features associated with a PCG signal are derived using S1, systole, S2, and diastole states of a PCG cycle.

In an embodiment, the extracted features associated with a PCG signal are obtained using time domain properties, frequency domain properties, energy properties, entropy properties and/or kurtosis properties.

In an embodiment, the extracted features include features corresponding to changes in frequency power of specific frequency bands of particular segments of the cardiac cycle.

In an embodiment, the machine learning component includes a classifier that may be implemented using a k-nearest neighbours algorithm (KNN), a support vector machine (SVM), and/or an artificial neural network (ANN) that may include a convolutional neural network (CNN).

In accordance with a fourth aspect of the present invention, there is provided a method of monitoring an animal body for a medical anomaly, the method comprising:
providing a plurality of phonocardiogram sensing devices, each phonocardiogram sensing device including:
   a body audio sensing device including a body audio sensing transducer arranged to sense animal body audio when the body audio sensing device is disposed adjacent an animal body in a body audio sensing position, wherein an acoustic path for body audio is defined between the animal body and the body audio sensing transducer when the body audio sensing device is disposed in the body audio sensing position; and
   an ambient audio sensing device including an ambient audio sensing transducer arranged to sense ambient audio present in an environment adjacent the phonocardiogram sensing device when the body audio sensing
device is disposed in the body audio sensing position;
disposing the body audio sensing devices at defined locations on the animal body;
using the body audio sensing devices to produce body audio signals indicative of animal body audio and ambient audio signals indicative of the ambient audio present in the environment adjacent each body audio sensing device;
using the ambient audio signals to remove ambient associated noise from the body audio signals and thereby produce noise processed body audio signals having an increased signal to noise ratio;
extracting a plurality of features from the noise processed body audio signals and producing at least one feature vector comprising a plurality of features; and
using the at least one feature vector to predict at least one medical anomaly associated with the animal body.

### BRIEF DESCRIPTION OF THE DRAWINGS

The present invention will now be described, by way of example only, with reference to the accompanying drawings, in which:

FIG. 1 is a diagrammatic perspective view of a phonocardiogram device in accordance with an embodiment of the present invention;

FIG. 2 is a diagrammatic perspective view of a diaphragm of the phonocardiogram device shown in FIG. 1;

FIG. 3 is a diagrammatic cross-sectional view of the phonocardiogram device shown in FIG. 1;

FIG. 4 is a diagrammatic cross-sectional view of a vibration reducing connection of the phonocardiogram device shown in FIG. 1;

FIG. 5 is a diagrammatic cross-sectional view of a portion of the phonocardiogram device shown in FIG. 3;

FIG. 6 is a block diagram of functional components of the phonocardiogram device;

FIG. 7 is a block diagram of functional components of a monitoring system according to an embodiment of the present invention;

FIG. 8 is a representation of a front portion of a wearable garment according to an embodiment of the present invention, the wearable garment including a plurality of the phonocardiogram devices shown in FIGS. 1 to 6 and a plurality of ECG electrodes;

FIG. 9 is a representation of a rear portion of the wearable garment shown in FIG. 8;

FIG. 10 is a representation of a neck portion of the wearable garment shown in FIGS. 8 and 9;

FIG. 11 is a block diagram of components of a filtering stage of the monitoring system shown in FIG. 7;

FIG. 12 is a block diagram of components of a signal conditioner of the monitoring system shown in FIG. 7;

FIG. 13 is a block diagram of a data processor filtering stage of the monitoring system shown in FIG. 7;

FIG. 14 is a flow diagram illustrating steps of a method of monitoring an animal body according to an embodiment of the present invention;

FIG. 15 show plots of phonocardiogram signals of a normal subject and a subject with cardiovascular disease (CVD); and

FIG. 16 is a block diagram of functional components of an alternative monitoring system according to a further embodiment of the present invention.

### DESCRIPTION OF AN EMBODIMENT OF THE INVENTION

While the present embodiments are described in relation to a human patient body, it will be understood that the present invention is equally applicable to other animal bodies.

It will also be understood that while the present embodiments are described in relation to heart and lung anomalies, the invention may equally be applied to other medical anomalies, the important aspect being the medical anomaly is associated with at least body generated audio signals and the medical anomaly is detectable based on the body generated audio signals.

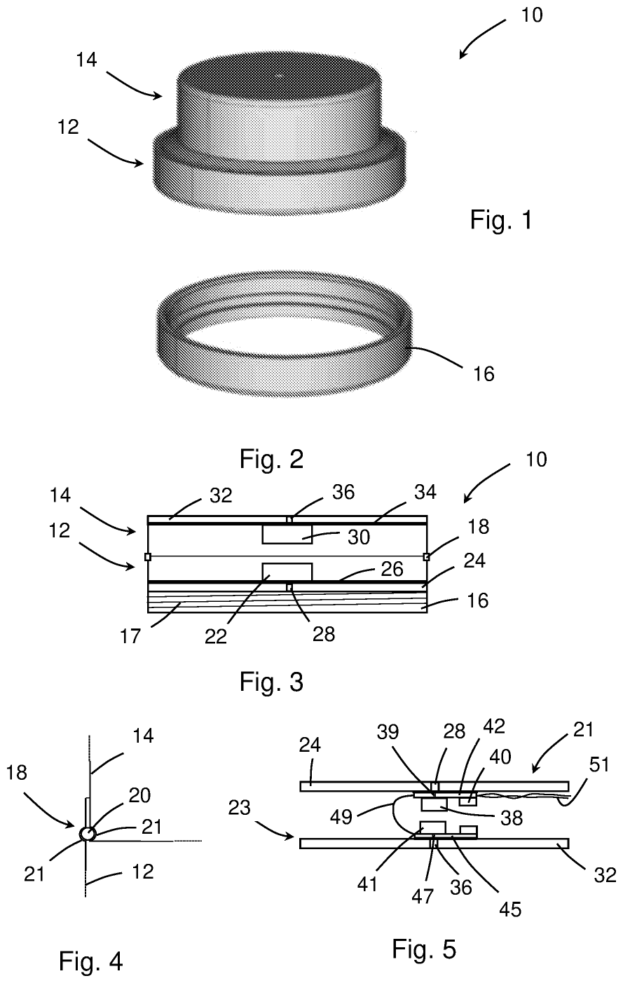

Referring to the drawings, FIGS. 1 and 2 show an example phonocardiogram (PCG) device 10 according to an embodiment of the present invention.

The PCG device 10 includes a body audio sensing device 12 and an ambient audio sensing device 14 connected to the body audio sensing device 12. During use, the PCG device 10 is disposed relative to a patient such that the body audio sensing device 12 contacts the patient's skin. The body audio sensing device 12 includes a diaphragm 16, as shown in FIG. 2, for example formed of flexible plastics material, that defines an audio cavity when the PCG device 10 is disposed adjacent a patient's skin during use. The diaphragm 16 serves to enhance body sounds emanating from the patient.

As shown more particularly in FIG. 3, in this example the body audio sensing device 12 is connected to the ambient audio sensing device 14 using a vibration reducing connection 18. In an example, as shown in FIG. 4, the vibration reducing connection 18 includes a resilient O-ring 20 that is received in opposed circumferential grooves 21 when the body audio sensing device 12 is connected to the ambient audio sensing device 14. In this way, transfer of vibrations between the body audio sensing device 12 and the ambient audio sensing device 14 is reduced.

The body audio sensing device 12 includes a body audio sensing component 22 attached to a body audio wall 24, for example using an adhesive layer 26. A body audio aperture 28 is formed in the body audio wall 24 so that audio associated with the patient's body during use is able to pass from the cavity defined by the diaphragm 16 to the body audio sensing component 22. In this example, the diaphragm 16 is provided with an internal screw threaded portion 17 that facilitates secure connection of the diaphragm 16 to the body audio wall 24.

Similarly, the ambient audio sensing device 14 includes an ambient audio sensing component 30 attached to an ambient audio wall 32, for example using an adhesive layer 34. An ambient audio aperture 36 is formed in the ambient audio wall 32 so that ambient audio is able to pass from the surrounding environment, to the ambient audio sensing component 30.

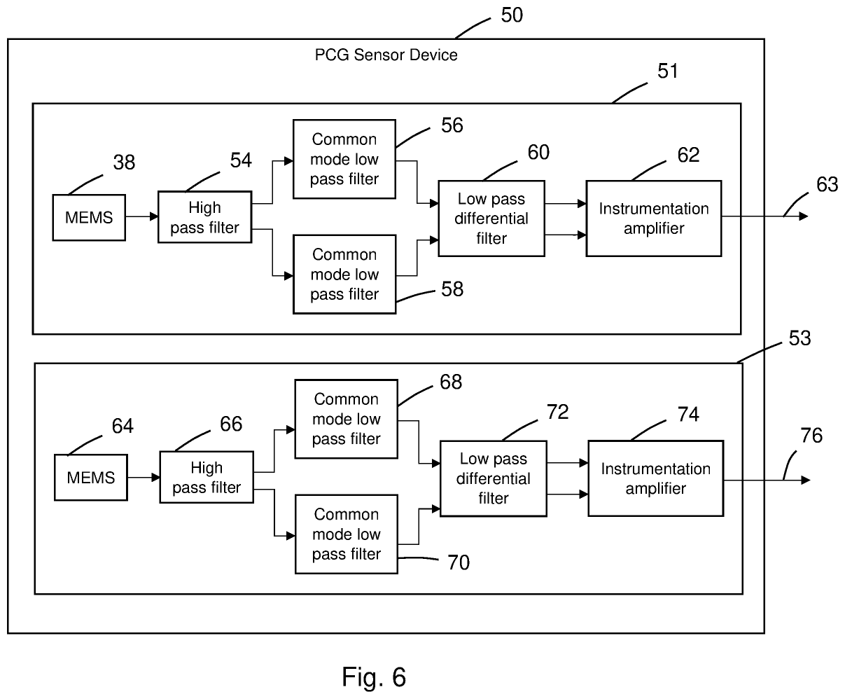

FIG. 5 illustrates a portion of the phonocardiogram device 12, and in particular shows components 21 of the body audio sensing component 22 and components 23 of the ambient audio sensing component 30.

As shown, the body audio sensing component 22 includes a body audio sensing transducer 38 and an audio processing device 40 mounted on a circuit board 42. In this example, the body audio sensing transducer 38 is a micro-electromechanical system (MEMS) microphone, although it will be understood that any suitable electromechanical transducer is envisaged. The circuit board 42 is provided with an aperture 39 that aligns with the body audio aperture 28 and a sensing portion of the body audio sensing transducer 38.

Similarly, the ambient audio sensing component 30 includes an ambient audio sensing transducer 41 and an audio processing device 43 mounted on a circuit board 45. In this example, the ambient audio sensing transducer 41 is a micro-electromechanical system (MEMS) microphone, although it will be understood that any suitable electromechanical transducer is envisaged. The circuit board 45 is provided with an aperture 47 that aligns with the ambient audio aperture 36 and a sensing portion of the ambient audio sensing transducer 41.

In this example, the respective circuit boards 42, 45 of the body audio sensing component 22 and the ambient audio sensing component 30 are electrically connected together using a flexible band connector 49 so that a common communications path 51 for both the body audio sensing component 22 and the ambient audio sensing component 30 is provided.

Example functional components 50 of the PCG device 10 are shown in FIG. 6. The functional components 50 include body audio functional components 51 and ambient audio functional components 53.

The body audio functional components 51 include the body audio sensing transducer 38, which in this example is a MEMS microphone, and a high pass filter 54 arranged to filter out low frequency signals from a transducer signal produced by the MEMS microphone 38. In the present example, the high pass filter 54 is a single pole RC high pass filter with a 3 dB cut off at 8.5 Hz, although it will be understood that any other suitable high pass filter is envisaged.

The body audio functional components 51 also include a pair of single pole common mode low pass filters 56, 58 disposed at respective inputs of a low pass differential filter 60 that serves to minimise amplification of common mode signals. Outputs of the low pass differential filter 60 are connected to inputs of a low noise differential instrumentation amplifier 62 that provides a high common mode rejection ratio and a very high differential input impedance. The common mode low pass filters 56, 58 serve to reduce RF rectification in the low noise instrumentation amplifier 62, and in this example each common mode low pass filter 56, 58 is arranged to provide a 3 dB cut off at 40 kHz. In this example, the low pass differential filter 60 is arranged to provide a 3 db cut off at 1.9 kHz. However, it will be understood that any other suitable common mode low pass filters are envisaged, and any other suitable low pass differential filter is envisaged. In this example, the low noise instrumentation amplifier 62 provides a gain of about 5, although it will be understood that any other suitable gain is envisaged.

The ambient audio functional components 53 of the ambient audio sensing device 14 are similar to the body audio functional components 51 of the body audio sensing device 12 in that the ambient audio functional components 53 include an ambient audio sensing transducer 64, which in this example is a MEMS microphone, a high pass filter 66 of similar configuration to the high pass filter 54, a pair of single pole common mode low pass filters 68, 70 of similar configuration to the pair of single pole common mode low pass filters 56, 58, a low pass differential filter 72 of similar configuration to the low pass differential filter 60, and a low noise instrumentation amplifier 74 of similar configuration to the low noise instrumentation amplifier 62 except that the gain of the instrumentation amplifier 74 is about 20 instead of 5.

The body audio sensing device 12 produces a body audio signal 63 indicative of sensed body audio, and the ambient audio sensing device 14 produces an ambient audio signal 76 indicative of sensed ambient audio. The ambient audio signal 76 is used to remove ambient associated noise from the body audio signal 63 and thereby increase the signal-to-noise ratio (SNR) of the body audio signal 63.

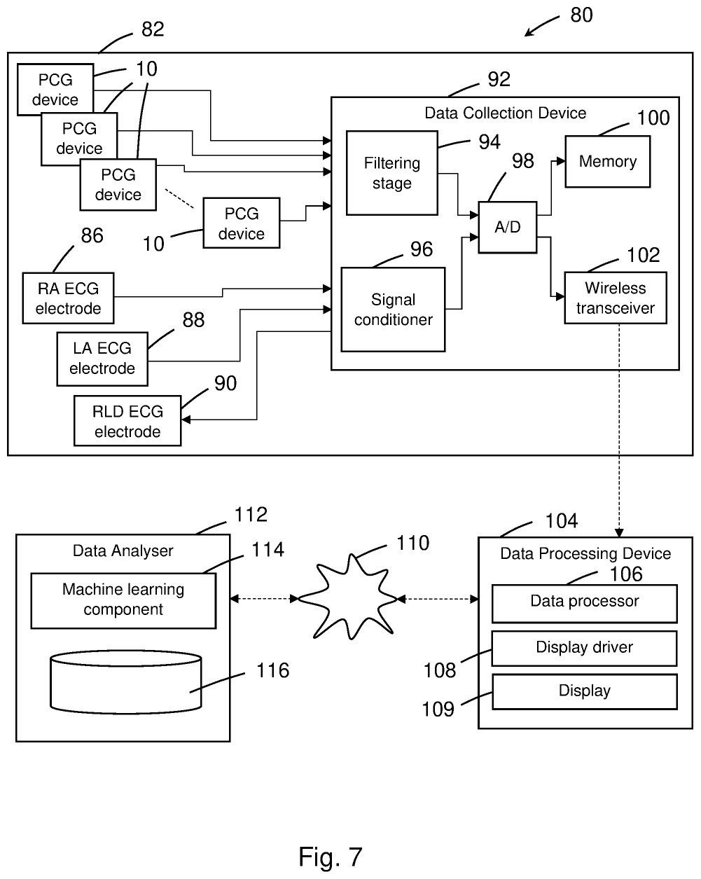

FIG. 7 illustrates an example monitoring system 80 for monitoring body generated signals, in particular body generated audio signals and body generated electrical signals. In this embodiment, the monitoring system 80 includes a wearable garment 82, in this example in the form of a vest, several PCG devices 10, in this example 13 PCG devices 10, and several ECG electrodes, in this example a right atria (RA) electrode 86, a left atria (LA) electrode 88 and a right leg drive (RLD) electrode 90.

In this example, the monitoring system 80 is arranged to monitor heart-related audio and electrical signals, and lung-related audio signals, although it will be understood that other types of body generated signals may in addition or alternatively be monitored.

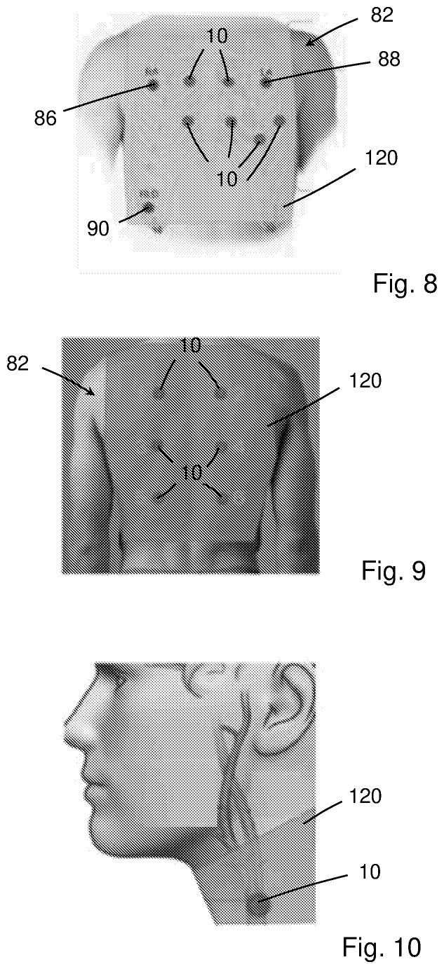

The PCG devices 10 and ECG electrodes 86, 88, 90 are mounted on the vest 82 at locations selected to optimise collection of relevant body generated signals. An example wearable garment 82 is shown in FIGS. 8 to 10. It will be appreciated that the vest 82 ensures accurate positioning of the PCG devices 10 and ECG electrodes 86, 88, 90 relative to the patient's body, and also ensures that an even, consistent pressure exists between the patient's body and the PCG devices 10 and ECG electrodes 86. 88. 90.

FIG. 8 illustrates a front portion of the vest 82 with locations of PCG devices 10 and ECG electrodes 86, 88, 90 indicated. In this example, 6 PCG devices are disposed on the front portion with 2 PCG devices 10 disposed on opposite sides of the patient's sternum in alignment with the patient's pulmonary artery, 2 PCG devices 10 disposed on opposite sides of the patient's sternum in alignment with the patient's tricuspid valve, 1 PCG device 10 disposed adjacent a mitral area on the midclavicular line, and 1 PCG device 10 disposed adjacent a midaxillary area on the midaxillary line.

In this example, the RA ECG electrode 86 is disposed rightwardly of the patient's atria, and the LA ECG electrode 88 is disposed leftwardly of the patient's atria. The RLD ECG electrode 90 is disposed vertically in alignment with the RA ECG electrode 86 at a location well below the patient's heart.

FIG. 9 illustrates a rear portion of the vest 82 with locations of PCG devices 10 indicated. In this example, 6 PCG devices 10 are disposed on the rear portion with 2 PCG devices 10 disposed on opposite sides of a vertical centreline just below the patient's scapula, 2 PCG devices 10 disposed on opposite sides of the vertical centreline at a middle portion of the patient's back, and 2 PCG devices 10 disposed on opposite sides of the vertical centreline adjacent lower lobes of the patient's lungs. The 6 PCG devices 10 disposed on the rear portion of the vest 82 are arranged to monitor sounds produced by the patient's lungs.

The vest 82 also includes a neck PCG device 10 disposed at a patient's neck area, as shown in FIG. 10. The neck PCG device 10 is disposed at a location to sense audio from the patient's carotid artery.

The PCG devices 10 and ECG electrodes 86, 88, 90 collectively provide body audio and body electrical signals that are usable to detect and characterise body anomalies, in this example heart and lung anomalies.

As illustrated in FIG. 7, in this example the vest 82 also includes a data collection device 92 that synchronously receives signals from the PCG devices 10 and the ECG electrodes 86, 88, 90. Synchronously receiving data enables data processing components to map time dependency and spatial dependency.

The data collection device 92 includes a filtering stage 94, a signal conditioner 96 and an analogue to digital (A/D) converter 98. A rechargeable power storage device (not shown) may also be provided for supplying electrical power to operative components of the vest 82.

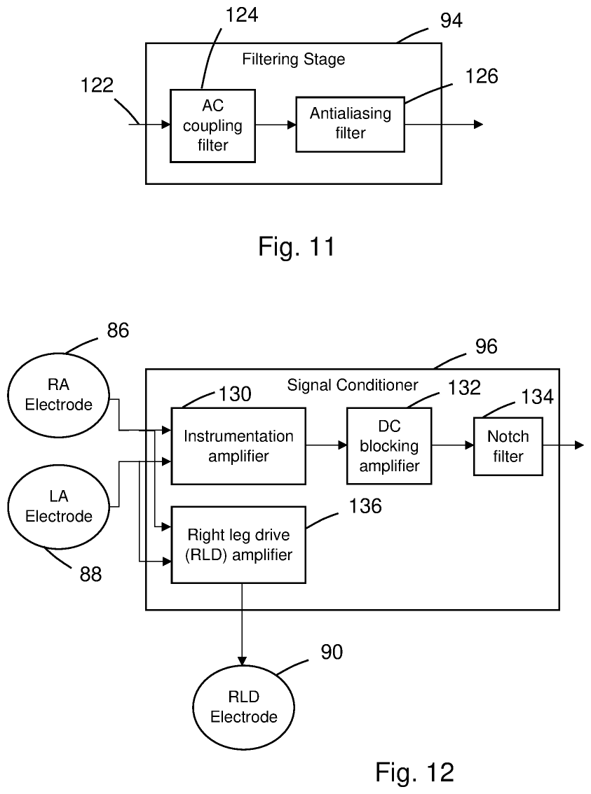

The filtering stage 94 receives data from the PCG devices 10. Functional components of the filtering stage 94 are shown in FIG. 11.

The filtering stage 94 includes an AC coupling filter 124 arranged to receive body audio signals 122 produced by the PCG devices 10 and remove any DC offset voltage that may have been produced by the instrumentation amplifier 62. In this example, the AC coupling filter 124 is a high pass single pole RC filter with a 3 dB cut off at 34 Hz, although it will be understood that suitable variations are envisaged.

The filtering stage 94 also includes an antialiasing filter 126 which ensures that no significant aliasing occurs in the A/D converter 98. In this example, the antialiasing filter 126 is a low pass single pole filter with a 3 dB cut off at 8 kHz, although it will be understood that any suitable variations are envisaged.

In this example, the A/D converter 98 is a delta-sigma 24-bit type A/D converter that samples all input signals simultaneously at a clock frequency of about 8 MHz and includes a digital decimation filter arranged to operate as a linear phase, finite impulse response third order low pass filter with a 3 dB cut off at 4 kHz, although it will be understood that any suitable variations are envisaged.

The signal conditioner 96 receives data from the ECG electrodes 86, 88, 90. Functional components of the signal conditioner 96 are shown in FIG. 12.

The signal conditioner 96 includes an instrumentation amplifier 130 that receives ECG signals from the RA and LA ECG electrodes 86, 88 and amplifies the difference between them. In this example, the instrumentation amplifier 130 is a high gain differential instrumentation amplifier with a gain of about 100, although it will be understood that any suitable variations are envisaged.

The signal conditioner 96 also includes a DC blocking amplifier 132 arranged to remove DC components from the amplified signal produced by the instrumentation amplifier 130, and a notch filter 134, in this example arranged to suppress interference at about 50 Hz.

The signal conditioner 96 also includes a right leg drive (RLD) amplifier 136 that extracts a common mode signal from signals associated with the RA and LA electrodes 86, 88, inverts the common mode signal, and uses the inverted signal to drive a feedback electrical signal into the patient at the RLD electrode 90. In this way, common mode rejection is improved since the injected signal counteracts the common mode signal that would otherwise be present in the RA and LA electrodes 86, 88.

The digitised signals produced by the A/D converter 98 and that are indicative of the sensed body audio and electrical signals are stored in a memory 100, and the digitised signals are sent either from the memory 100 or substantially in real time to a data processing device 104 using a wireless transceiver 102. In the present example, the digitised signals are wirelessly sent using a Bluetooth or WiFi protocol, although it will be understood that any suitable communications protocol may be used.

The data processing device 104 carries out further signal processing, noise cancellation processing, and analysis of the body audio and electrical signals to extract features that will be used to determine whether any patient anomalies exist, in this example heart and lung anomalies. The data processing device 104 also serves to display results of the audio and electrical signal analysis to a user.

In this example, the data processing device 104 includes a data processor 106 and a display driver 108 arranged to drive a display 109. In this example, the data processing device 104 is implemented using any suitable computing device that may be a desktop computer, laptop computer, tablet computer or smartphone.

The data processing device 104 also includes suitable software to implement a user interface for the monitoring system 80, in particular so that the user can control operations of the monitoring system 80, including capture of audio and electrical body signal by the PCG devices 10 and ECG electrodes 86, 88, 90, commencement of data analysis of the captured signals, viewing and analysis of the diagnosis results produced by the data analyser 112, and analysis of historical results including progression of disease.

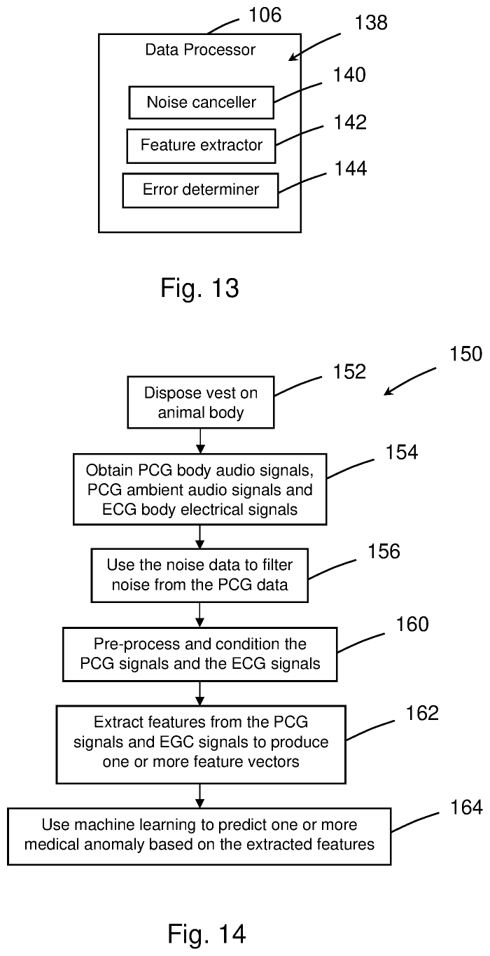

Functional components 138 of the data processor 106 are shown in FIG. 13. As shown, the functional components 138 include a noise canceller 140 that removes a noise signal from the body audio signal 63 using the ambient audio signal 76 to improve the signal-to-noise ratio (SNR) of the body audio signals.

The PCG device 10 and associated monitoring system 80 are capable of detecting audio over a wide frequency range, in this example between 10 Hz and 5 kHz, and it will be understood that the working frequency range of the PCG monitoring system 80 is made possible by improving the SNR of the body audio signal 63.

The inventors recognise that in excess of 200 different features exist for detecting various heart conditions, although it is impractical to use them all to make determinations in relation to existence of medical anomalies. For this reason, the functional components 138 also include a feature extractor 142 arranged to extract features from the processed PCG and ECG signals, and produce feature vectors usable as inputs to a machine learning algorithm trained to predict patient anomalies using the feature vectors.

The functional components 138 also include an error determiner 144 arranged to remove PCG and/or ECG signals that are considered to be anomalous.

The feature vectors may include features synchronously derived from one or both of the ECG signals and PCG signals.

The arrangement is such that each feature vector is mapped to a particular medical anomaly, such as a particular type of cardiovascular disease (CVD), and the features included in each feature vector are selected using a statistical gain ranking method such as information gain ratio (IGR). The features used to predict patient anomalies are typically based on repeatable patterns in the ECG and PCG signals associated with the heart rate.

Example features associated with the ECG signals are derived from the R peaks, P peaks, Q points, S-points and T-peaks, and may include a time interval between R peaks, a difference between successive R peak time intervals, and a time between P and R peaks.

The R, P, Q, S and T related features may be characterised using:
frequency domain properties that are obtained using a fast Fourier transform (FFT) algorithm, for example average, variance and higher order moments, skewness, kurtosis, and spectral entropy which is indicative of the peakiness of the spectrum; and
time domain and time-frequency domain properties including wavelets or wavelet packets (WP) and synchronised wavelet packets (SWP).

A wavelet may be derived from the R peak of the ECG signal, and various statistical energy measures may be calculated based on wavelet coefficients that are associated with the shape of the R wave. WP or SWP are similar, except that they are associated with the full ECG signal. Typically, the fourth level of a wavelet packet tree is used which corresponds to 16 coefficients. Similar statistical measures as for the FFT features may be used on the 16 coefficients.

Features associated with the PCG signals are usually related to a PCG cycle which can be segmented into four states-S1, systole, S2, and diastole. After segmentation of the PCG signal, time domain features, frequency domain features, energy features, entropy features and kurtosis are defined.

In a specific example, it has been observed that changes in the frequency power of specific frequency bands of particular segments of the cardiac cycle are indicative of coronary artery disease (CAD). Accordingly, by using appropriate band pass filters at between 3 and 9 bands, a set of features can be obtained that can be used to distinguish between non-CAD subjects and CAD subjects.

In particular, the inventors are aware that a frequency band between 20-30 Hz is useful for distinguishing non-CAD and CAD subjects.

The frequency band between 250-1000 Hz is also useful and the present arrangement whereby noise in the PCG signals is reduced using the ambient audio sensing component 30 enables this band to be used to obtain relevant features that are indicative of patient anomalies. It will be understood that by suppressing external noise, higher frequency heart related sounds can be enhanced.

However, alternative methodologies for determining features from the PCG signals are envisaged. For example, the full PCG signal may be analysed by analysing wavelets/wavelet packets (WP) and/or synchronised wavelet packets (SWP) associated with the PCG signal.

Table 1 below illustrates example features obtainable from the body PCG and ECG signals that may be used in feature vectors. However, it will be understood that other features and feature vectors may be used.

TABLE 1

| Features | Feature Vector |
|---|---|
| 1. Time domain features (TDFs): | TDFs = [ZCC, VTC, FF] |
| Zero-crossing counts (ZCC) | Measures the signal activity |
| Variance of the turns count (VTC) | and signal complexity |
| Form factor (FF) | |
| 2. Sub-band moments (SBMs): | SBMs = [Sμ_{1, 2 . . . Tn}, Sσ_{1, 2 . . . Tn}, |
| Sub-band mean (S_{μ}) | Ss′_{1, 2 . . . Tn}, SK′_{1, 2 . . . Tn}] |
| Sub-band standard deviation (S_{σ}) | Where Tn is the total number of |
| Sub-band skewness (S′ₛ) | sub-bands |
| Sub-band kurtosis (S′_{K}) | Measures the sub-band |
| | wise spectrum distribution |
| 3. Sub-band spectral entropy (SSE) | SSE = [Slm_{1, 2 . . . Tn}] |
| | Measures the sub-band |
| | wise spectrum randomness |
| 4. Moments of PSD function (MPF) | MPF = [_{fm}, f_{std}, f_{skew}, fₖᵤᵣₜ] |
| Mean frequency (fₘ) | Measures the concentration |
| Standard deviation of the frequency | of signal power, spectrum |
| (f_{std}) | spread, spectrum |
| Skewness of the frequency (f_{skew}) | symmetry/asymmetry, and |
| Kurtosis of the frequency (fₖᵤᵣₜ) | long-tailed function |
| | respectively |
| 5. Power ratio (PR) | Measures the band of |
| | interest |

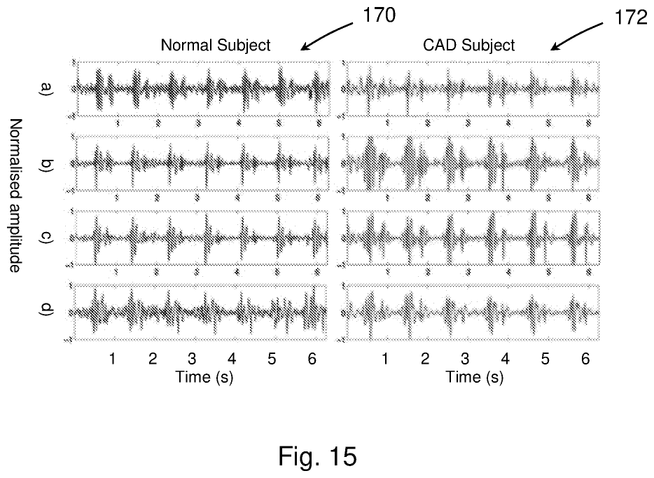

FIG. 15 shows example waveforms representing PCG signals obtained from 4 locations on a patient—a) pulmonary location, b) tricuspid location, c) mitral location and d) midaxillary location—for a normal subject 170 and a subject with coronary artery disease (CAD) 172.

Example medical anomalies that may be related to one or more feature vectors include:

### Heart

valvular disorders;
heart failure;
cardiac arrhythmias including atrial fibrillation, atrial flutter,
supraventricular tachycardia, ventricular fibrillation and ventricular tachycardia

### Lungs

air and fluid in the lungs due to infectious and non-infectious diseases;
structural changes in the thoracic cavity;
pneumonia;
pleural effusion;
pneumothorax;
chronic obstructive pulmonary disease (COPD);
asthma

The feature vectors produced by the feature extractor 142 are communicated to a data analyser 112 that in this example is disposed remotely of the data processing device 104 and accessed through a wide area network such as the Internet 110.

The data analyser includes a machine learning component 114 arranged to produce predictions in relation to medical anomalies using input feature vectors, and a data storage device 116 for storing the feature vectors and the diagnosis predictions. Storing the feature vectors and the diagnosis predictions enables disease progression to be monitored over time. The data storage device 116 may also be arranged to store the processed audio and electrical body generated signals, for example the audio and electrical body generated signals that are received by the data processing device 104 from the vest 82.

The machine learning component may include any suitable machine learning arrangement that may be hardware and/or software based, and the machine learning component may be of any suitable type, including a classifier implemented using a k-nearest neighbours algorithm (KNN), a support vector machine (SVM), and/or an artificial neural network (ANN) that may include a convolutional neural network (CNN).

The machine learning component may be trained in any suitable way, for example using supervised learning to establish relationships between defined input feature vectors and medical anomalies.

FIG. 14 illustrates a method 150 of monitoring an animal body according to an embodiment of the present invention, the method using the monitoring system 80 shown in FIG. 7.

During use, a user, such as a clinician, first disposes the vest 82 on a patient and uses the data processing device 104, in this example dedicated software implemented on the data processing device 104, to synchronously capture body audio and ambient audio signals using the PCG devices, and ECG signals using the ECG electrodes, as indicated at steps 152 and 154. As indicated at step 156, the processed ambient audio signals are then used to remove ambient associated noise from the body audio signals to improve the signal-to-noise ratio of the body audio signals. The captured signals are then processed by filtering and conditioned to reduce noise and prepare the signals for analysis, as indicated at step 160. As indicated at step 162, features that are relevant to diagnosis medical conditions are extracted from the processed noise-reduced body audio and body electrical signals, and the extracted features are then used to produce feature vectors that are used as inputs to a machine learning component trained to recognise relationships between the feature vectors and medical anomalies.

It will be appreciated that the present invention facilitates early diagnosis of medical anomalies, in particular heart and lung diseases, and patients suspected of CVD and/or lung disease can be identified without the need to rely on specialist skills of a clinician.

It will also be appreciated that using multiple body sensors, in particular multiple PCG sensing devices, significantly enhances the depth of data that is available for diagnosing medical anomalies, in particular heart and lung anomalies, and as a consequence the granularity of diagnosis possible with the present invention is greater than corresponding diagnosis arrangements known hitherto.

It will also be appreciated that the results obtainable by the present invention are more extensive than is possible by conventional auscultation by a clinician since the frequency range covered is wider than the typical human hearing range. In particular, the present invention is arranged to analyse relatively low frequencies that are relevant to diagnosis of specific medical anomalies but not audible by a human.

It will also be appreciated that since the results produced by the monitoring system are progressively stored, it is possible to monitor disease progression over time.

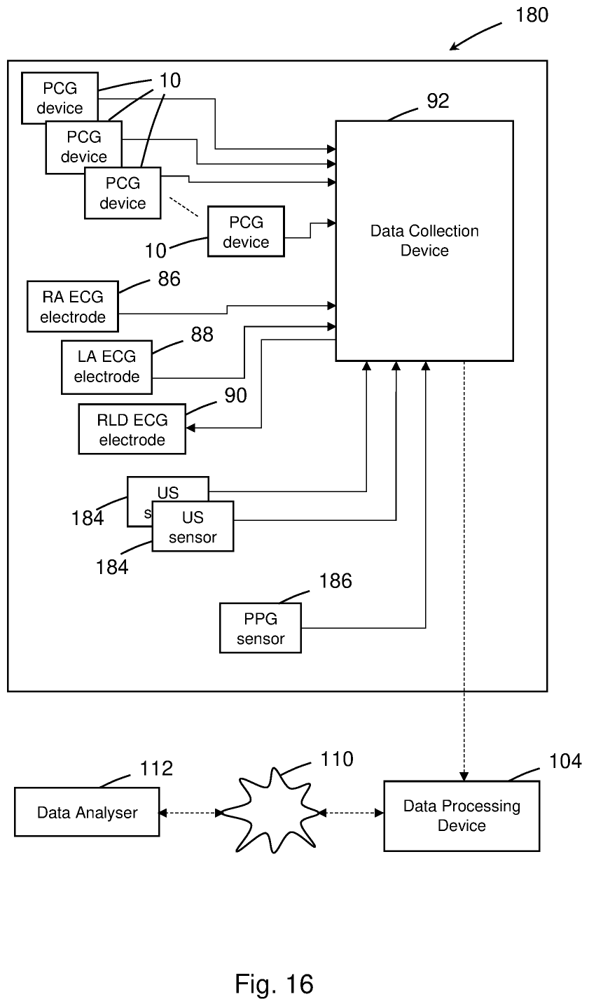

Functional components of a monitoring system 180 according to a further embodiment of the present invention are shown in FIG. 16. Like and similar features are indicated with like reference numerals.

The monitoring system 160 is similar to the monitoring system 80 shown in FIG. 7 in that multiple PCG devices 10 and multiple ECG electrodes 86, 88, 90 are included in a wearable garment 182. In the present embodiment, in addition to the PCG devices 10 and ECG electrodes 86, 88, 90, the monitoring system 180 also includes at least one ultrasound sensor 184, in this example also incorporated into the wearable garment 182, and at least one photoplethysmography (PPG) sensor 186, in this example also incorporated into the wearable garment 182.

The ultrasound sensor(s) 184 provide information that can be used to obtain respiratory and heart cycle data. For example, the ultrasound sensor(s) 184 may be disposed during use adjacent a patient's heart or thorax, and the information assessed simultaneously with information from the PPG and PCG sensors 10, 186 and the ECG electrodes 86, 88, 90 to obtain an indication of Left Ventricle Ejection fraction (LVEF) by constructing a feature vector from the multiple sources of information and applying trained machine learning to the feature vector. LFEV data can be used to obtain an indication of heart failure or cardiomyopathy. Ejection fraction is a measure of the amount of blood in the heart compared to the amount of blood pumped out of the heart, and therefore the ejection fraction is indicative of how efficiently the heart is pumping blood to the body.

The photoplethysmography (PPG) sensor 186 can be used to monitor changes in blood volume and blood oxygenation, and therefore using a PPG sensor 186 it is possible to monitor pulsatile arterial blood flow and obtain an associated photoplethysmogram waveform that can be used to derive physiological parameters including heart rate variability, blood pressure, ankle-brachial pressure, cardiovascular disease, aging, neurological disorder, lung disease and respiratory rate. The PPG sensor 186 may be of a type arranged to measure the amount of light absorbed or reflected by tissue.

The data obtained from the ultrasound sensor(s) 184 may also be combined with information derived from the PPG sensor 186 to detect the presence of fluid in the lungs.

In the claims that follow and in the preceding description of the invention, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention.

It is to be understood that, if any prior art publication is referred to herein, such reference does not constitute an admission that the publication forms a part of the common general knowledge in the art, in Australia or any other country.

Modifications and variations as would be apparent to a skilled addressee are deemed to be within the scope of the present invention.

1. A system for monitoring body generated data, the system comprising:
a wearable garment comprising:
at least one phonocardiogram (PCG) sensing device disposed at a location selected to optimise collection of relevant body audio signals, the PCG sensing device including:
a body audio sensing device including a body audio sensing transducer arranged to sense animal body audio when the body audio sensing device is disposed adjacent an animal body in a body audio sensing position, wherein an acoustic path for body audio is defined between the animal body and the body audio sensing transducer when the body audio sensing device is disposed in the body audio sensing position; and
an ambient audio sensing device including an ambient audio sensing transducer arranged to sense ambient audio present in an environment adjacent the phonocardiogram sensing device when the body audio sensing device is disposed in the body audio sensing position;
the body audio sensing device producing a body audio signal indicative of animal body audio, and the ambient audio sensing device producing an ambient audio signal indicative of the ambient audio, the ambient audio signal usable to increase the signal to noise ratio of the body audio signal; and
at least one ECG electrode disposed at a location selected to optimise collection of relevant body electrical signals;
a noise cancelling component arranged to use the ambient audio signal to remove ambient associated noise from the body audio signal and thereby produce a noise processed body audio signal having an increased signal to noise ratio; and
a feature extractor arranged to synchronously extract a plurality of features from the noise processed body audio signal and the body electrical signals and to use the synchronously extracted features to produce at least one feature vector comprising a plurality of features derived from the body audio signals and the body electrical signals; and
a data analysis component arranged to use the at least one feature vector to predict at least one medical anomaly associated with the animal body, the data analysis component comprising at least one machine learning component trained to learn relationships between the feature vectors and medical anomalies.

2-81. (canceled)

82. A system as claimed in claim 1, wherein the body audio sensing device includes any one or more of the following:
PCG filtering components arranged to filter body audio signals produced by the body audio sensing transducer; and/or
a PCG amplification component arranged to amplify body audio signals produced by the body audio sensing transducer.

83. A system as claimed in claim 1, wherein the ambient audio sensing device includes any one or more of the following:
ambient filtering components arranged to filter ambient audio signals produced by the ambient audio sensing transducer; and/or
an ambient amplification component arranged to amplify ambient audio signals produced by the ambient audio sensing transducer.

84. A system as claimed in claim 1, comprising a plurality of PCG sensing devices, the plurality of PCG sensing devices comprising:
a front plurality of PCG devices that includes PCG devices disposed during use on opposite sides of a sternum of the animal body in alignment with a pulmonary artery, PCG devices disposed during use on opposite sides of the sternum of the animal body in alignment with a tricuspid valve, a PCG device disposed during use adjacent a mitral area on the midclavicular line, and/or a PCG device disposed during use adjacent a midaxillary area on the midaxillary line; and/or
a rear plurality of PCG devices that includes PCG devices disposed on opposite sides of a vertical centreline just below a scapula of the animal body, PCG devices disposed on opposite sides of the vertical centreline at a middle portion of the back of the animal body, and/or PCG devices disposed on opposite sides of the vertical centreline adjacent lower lobes of the lungs of the animal body; and/or
at least one neck PCG device disposed at a patient's neck area to sense audio from the carotid artery.

85. A system as claimed in claim 1, wherein the at least one ECG electrode comprises:
a RA ECG electrode disposed rightwardly of an atria of the animal body;
a LA ECG electrode disposed leftwardly of the atria;
a RLD ECG electrode disposed vertically in alignment with the RA ECG electrode at a location below the heart of the animal body; and
the wearable garment includes a right leg drive (RLD) amplifier that uses signals obtained from the RA and LA electrodes to drive a feedback electrical signal into the animal body at the RLD electrode, the feedback signal serving to improve the common mode rejection by counteracting a common mode signal that would otherwise be present in the RA and LA electrodes.

86. A system as claimed in claim 1, comprising at least one ultrasound sensor for obtaining respiratory and heart cycle data.

87. A system as claimed in claim 86, wherein the at least one ultrasound sensor is disposed on the wearable device such that during use the at least one ultrasound sensor is disposed adjacent a patient's heart or thorax.

88. A system as claimed in claim 1, comprising at least one photoplethysmography (PPG) sensor for monitoring changes in blood volume and blood oxygenation.

89. A system as claimed in claim 88, wherein data from the at least one photoplethysmography (PPG) sensor is used to derive physiological parameters associated with heart rate variability, blood pressure, ankle-brachial pressure, cardiovascular disease, aging, neurological disorder, lung disease and/or respiratory rate.

90. A system as claimed in claim 88, comprising at least one ultrasound sensor, wherein data obtained from the at least one ultrasound sensor and data derived from the at least one PPG sensor are used to detect the presence of fluid in a patient's lungs.

91. A system as claimed in claim 1, comprising at least one ultrasound sensor and at least one PPG sensor, wherein signals from the at least one ultrasound sensor, the at least one PCG device, the at least one PPG sensor and/or the at least one ECG electrode are synchronously received.

92. A system as claimed in claim 1, wherein the wearable garment includes a wireless transceiver arranged to facilitate communication of data indicative of the PCG and ECG signals from the wearable device.

93. A system as claimed in claim 1, wherein the system is arranged to map time and spatial dependency of synchronously extracted features.

94. A system as claimed in claim 1, wherein the ECG signal includes R peaks, P peaks, Q points, S-points and T-peaks, and the feature vector includes features derived using a time interval between R peaks, a difference between successive R peak time intervals, and/or a time between P and R peaks.

95. A system as claimed in claim 1, wherein the feature vector includes:
features associated with an ECG signal that are obtained using frequency domain properties that may include average, variance and higher order moments; skewness; kurtosis;
and/or time domain and time-frequency domain properties that may be obtained using wavelets or wavelet packets (WP) and/or synchronised wavelet packets (SWP) of the ECG signal; and/or
features associated with a PCG signal that are derived using S1, systole, S2, and diastole states of a PCG cycle; and/or
features associated with a PCG signal that are obtained using time domain properties, frequency domain properties, energy properties, entropy properties and/or kurtosis properties; and/or
features corresponding to changes in frequency power of specific frequency bands of particular segments of the cardiac cycle.

96. A method of monitoring an animal body for a medical anomaly, the method comprising:
providing at least one phonocardiogram sensing device incorporated into a wearable garment, each phonocardiogram sensing device including:
a body audio sensing device including a body audio sensing transducer arranged to sense animal body audio when the body audio sensing device is disposed adjacent an animal body in a body audio sensing position, wherein an acoustic path for body audio is defined between the animal body and the body audio sensing transducer when the body audio sensing device is disposed in the body audio sensing position; and
an ambient audio sensing device including an ambient audio sensing transducer arranged to sense ambient audio present in an environment adjacent the phonocardiogram sensing device when the body audio sensing device is disposed in the body audio sensing position;
disposing each phonocardiogram sensing device at a location on the animal body selected to optimise collection of relevant body audio signals;
using each phonocardiogram sensing device to produce body audio signals indicative of animal body audio and ambient audio signals indicative of the ambient audio present in the environment adjacent each phonocardiogram sensing device;
using the ambient audio signals to remove ambient associated noise from the body audio signals and thereby produce noise processed body audio signals having an increased signal to noise ratio;
providing at least one ECG electrode on the wearable garment at a location selected to optimise collection of relevant body electrical signals.
synchronously extracting a plurality of features from the noise processed body audio signals and the body electrical signals and using the synchronously extracted features to produce at least one feature vector comprising a plurality of features derived from the body audio signals and the body electrical signals; and
using at least one machine learning component to predict at least one medical anomaly associated with the animal body the data analysis component using the at least one feature vector, the at least one machine learning component trained to learn relationships between the feature vectors and medical anomalies.

97. A method as claimed in claim 96, wherein the plurality of ECG electrodes comprises a RA ECG electrode disposed rightwardly of an atria of the animal body, a LA ECG electrode disposed leftwardly of the atria, and a RLD ECG electrode disposed vertically in alignment with the RA ECG electrode at a location below the heart of the animal body, and the method comprises using signals obtained from the RA and LA electrodes to drive a feedback electrical signal into the animal body at the RLD electrode, the feedback signal serving to improve the common mode rejection by counteracting a common mode signal that would otherwise be present in the RA and LA electrodes.

98. A method as claimed in claim 96, comprising using at least one ultrasound sensor to obtain respiratory and heart cycle data.

99. A method as claimed in claim 96, comprising using at least one photoplethysmography (PPG) sensor to obtain PPG data for monitoring changes in blood volume and blood oxygenation.

100. A method as claimed in claim 96, comprising using the PPG data to derive physiological parameters associated with heart rate variability, blood pressure, ankle-brachial pressure, cardiovascular disease, aging, neurological disorder, lung disease and/or respiratory rate.

101. A method as claimed in claim 100, comprising using data obtained from at least one ultrasound sensor and the PPG data to detect the presence of fluid in lungs of a patient.

102. A method as claimed in claim 96, comprising synchronously receiving signals from at least one ultrasound device, at least one PPG sensor, the PCG devices and/or the ECG electrodes.

103. A method as claimed in claim 96, comprising mapping time and spatial dependency of at least some synchronously extracted features.

104. A method as claimed in claim 96, wherein the ECG signal includes R peaks, P peaks, Q points, S-points and T-peaks of the ECG signal, and the feature vector includes features derived using a time interval between R peaks, a difference between successive R peak time intervals, and/or a time between P and R peaks.

105. A method as claimed in claim 96, wherein the feature vector includes:
features associated with an ECG signal using frequency domain properties that may include average, variance and higher order moments; skewness; kurtosis, and/or time domain and time-frequency domain properties that may be obtained using wavelets or wavelet packets (WP) and/or synchronised wavelet packets (SWP) of the ECG signal;
features associated with a PCG signal that are derived using S1, systole, S2, and diastole states of a PCG cycle;
features associated with a PCG signal that are obtained using time domain properties, frequency domain properties, energy properties, entropy properties and/or kurtosis properties; and/or
features corresponding to changes in frequency power of specific frequency bands of particular segments of the cardiac cycle.
